SEQUENCE LISTING (10 sequences)

SEQ ID NO: 1 - protein, 214 aa, Artificial Sequence [sequence omitted]

SEQ ID NO: 2 - protein, 122 aa, Artificial Sequence [sequence omitted]

SEQ ID NO: 3 - protein, 122 aa, Artificial Sequence
  Laboratory synthesis
QVQLVQSGGGVVQPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 4 - protein, 122 aa, Artificial Sequence
  Laboratory synthesis
QVQLVQSGGGVVQPGASVKVSCKASGYIFSNYWIQWVRQAPGQGLEWMGEILPGSGSTEYTENFKDRVTLTTDSSTTTAYMELKSLQFDDTAVYYCARYFFGSSPNWYFDVWGQGTLVTVSS

SEQ ID NO: 5 - protein, 456 aa, Artificial Sequence
  Laboratory synthesis
QVQLVQSGGGVVQPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGGGSGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 456 aa, Artificial Sequence
  Laboratory synthesis
QVQLVQSGGGVVQPGASVKVSCKASGYIFSNYWIQWVRQAPGQGLEWMGEILPGSGSTEYTENFKDRVTLTTDSSTTTAYMELKSLQFDDTAVYYCARYFFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGGGSGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 642 bp, Artificial Sequence
  Laboratory synthesis
gacatccagatgacccagagcccttcctctttaagcgccagcgtgggcgatagagtgaccatcacttgtggagccagcgagaacatttacggcgctttaaactggtaccagcagaagcccggcaaggcccccaagctgctgatctacggtgccaccaatctggccgatggcgtgccctctcgttttagcggcagcggatccggcaccgatttcactttaaccatcagctctttacagcccgaggatttcgccacctactactgccagaacgtgctgaacacacctttaacctttggccaaggtaccaaggtggagatcaagaggaccgtggccgcccccagcgtcttcattttccccccctccgacgagcagctgaagagcggcaccgccagcgtggtctgtctgctgaacaacttctaccctcgtgaggccaaggtgcagtggaaggtcgataatgctttacagagcggcaacagccaagaaagcgtgaccgagcaagattccaaggacagcacctactctttaagcagcactttaacactgagcaaggccgactacgagaagcacaaggtgtacgcttgcgaggtgacccatcaaggtttaagcagccccgtgaccaagtcttttaatcgtggcgagtgc

SEQ ID NO: 8 - DNA, 366 bp, Artificial Sequence
  Laboratory synthesis
caagttcagctggtgcagagcggagccgaggtgaagaagcccggtgccagcgtgaaggtgagctgcaaggccagcggctacatcttcagcaactactggatccagtgggtgagacaagctcccggtcaaggtttagagtggatgggcgagattttacccggtagcggcagcaccgagtacaccgagaacttcaaggatcgtgttactatgaccagagacaccagcacaagcacagtgtacatggagctgagctctttaaggagcgaggacaccgccgtgtactactgcgctcgttacttcttcggcagctcccccaactggtacttcgacgtgtggggccaaggtactttagttactgtgagcagc

SEQ ID NO: 9 - DNA, 366 bp, Artificial Sequence
  Laboratory synthesis
caagttcagctggtgcagagcggaggcggcgtggtgcagcccggtgccagcgtgaaggtgagctgcaaggccagcggctacatcttcagcaactactggatccagtgggtgagacaagctcccggtcaaggtttagagtggatgggcgagattttacccggtagcggcagcaccgagtacaccgagaacttcaaggatcgtgttactatgaccagagacaccagcacaagcacagtgtacatggagctgagctctttaaggagcgaggacaccgccgtgtactactgcgctcgttacttcttcggcagctcccccaactggtacttcgacgtgtggggccaaggtactttagttactgtgagcagc

SEQ ID NO: 10 - DNA, 366 bp, Artificial Sequence
  Laboratory synthesis
caagttcagctggtgcagagcggaggaggagtggtgcagcccggagctagcgtgaaggtgagctgcaaggccagcggctacatcttctccaactactggatccagtgggtgagacaagctcccggtcaaggtttagagtggatgggcgagattttacccggcagcggaagcaccgagtacaccgagaacttcaaggatcgtgttactttaaccaccgacagcagcacaaccaccgcctacatggagctgaagtctttacagttcgacgacaccgccgtgtactactgcgctcgttacttcttcggcagcagccccaactggtacttcgacgtgtggggccaaggtacattagttactgtgagcagc

(19) United States
(12) Patent Application Publication
(10) Pub. No.: US 2024/0067745 A1
(43) Pub. Date: Feb. 29, 2024

(54) Anti-C5 humanized monoclonal antibody with low immunogenicity and low ADCC/CDC function and its application

(71) Applicant: ABMAX BIOTECHNOLOGY CO. LTD., Beijing (CN)

(72) Inventors: Le SUN, Beijing (CN); Maohua LI, Beijing (CN); Wenlin REN, Beijing (CN)

(21) Appl. No.: 17/761,154

(22) PCT Filed: Jul. 9, 2020
(86) PCT No.: PCT/CN2020/100981

§ 371 Date: Nov. 15, 2023

(30) Foreign Application Priority Data
Jul. 11, 2019 (CN) 201910623477.8

(51) Int. Cl. C07K 16/28; A61P 13/12

(52) U.S. Cl. CPC C07K 16/2896; A61P 13/12; C07K 2317/24; C07K 2317/524; C07K 2317/565; C07K 2317/71

(57) ABSTRACT

The invention provides an anti-C5 humanized monoclonal antibody with low immunogenicity and low ADCC/CDC function and its application. By modifying the amino acid sequence of the framework region of Eculizumab monoclonal antibody, the immunogenicity was reduced, and the antibody was replaced from IgG2 subtype to IgG1 subtype, and a flexible amino acid sequence was inserted between the CDR3 and CH2 regions of the heavy chain of the IgG1 antibody to reduce the immunogenicity. The purpose of ADCC/CDC function is to improve the stability of the antibody and prolong its half-life. The binding affinity of the monoclonal antibody of the invention to human C5 is similar to that of the original Eculizumab antibody, it can specifically block the complement hemolytic activity of C5 and the production of C5a and can be used for the preparation of C5-targeted paroxysmal nocturnal hemoglobinuria and atypical the drug for the treatment of hemolytic uremic syndrome has excellent clinical therapeutic value.

### CROSS REFERENCE

This application claims the priority of Chinese Patent Application No. 201910623477.8 filed on Jul. 11, 2019, with the patent title of “Anti-C5 Humanized Monoclonal Antibody with Low Immunogenicity and Low ADCC/CDC Function and Its Application”, which is fully disclosed The contents are incorporated herein by reference in their entirety.

### TECHNICAL FIELD

The present invention relates to the preparation and application of therapeutic engineering antibodies, and mainly relates to a monoclonal antibody with low immunogenicity and low ADCC/CDC function aimed at human C5 target and its application.

### BACKGROUND TECHNIQUE

Eculizumab can specifically bind to human terminal complement protein C5 and block the release of inflammatory factor C5a and the formation of C5b-9 by inhibiting the cleavage of human complement C5 to C5a and C5b. C5 is a complement component that plays an important role in inflammatory responses and plays an important role in innate immune defense, but overactivation of complement can lead to severe tissue damage. C5a is one of the important products of complement activation and is involved in the occurrence and development of many inflammatory and autoimmune diseases such as sepsis, sepsis, acute lung injury, allergy, and asthma. Using C5a monoclonal antibody to block the signaling pathway can effectively reduce the inflammatory response, providing a new idea for the treatment of inflammatory and autoimmune diseases. The precursor protein C5 is cleaved into two fragments, C5a and C5b under the action of C5 convertase. The complement system activation product C5a is an anaphylatoxin, an important mediator and chemokine of inflammatory response; C5b is involved in the formation of membrane attack complexes. C5a monoclonal antibody has three major application areas: one is to inhibit the body's acute response, acute inflammation, such as acute lung injury; the other is to treat chronic autoimmune diseases, such as HS (hidradenitis suppurativa). The “drug” adalimumab, which sold $18.2 billion in 2017, is 10% effective, while the C5a monoclonal antibody is more than 80% effective; third, it can be used in combination with PD-1 to treat tumors.

Eculizumab monoclonal antibody is highly immunogenic in the host, and up to 23% of patients will develop an immune response with this monoclonal antibody, which may cause immune complex-mediated antibody or fragment clearance from the circulation and cause repeated Administration is inappropriate for therapy, thereby reducing therapeutic benefit to the patient and limiting re-administration of the antibody. Based on this, if the immunogenicity of antibody drugs can be reduced by replacing highly immunogenic non-antigen-binding sites in antibodies with low-immunogenic homologous sequences without affecting antibody affinity and specificity, on the one hand, the safety of the monoclonal antibody can be improved; on the other hand, it can increase the half-life of the drug, which can also improve the efficacy while reducing its dosage. At the same time, due to the problem of its immunogenicity, Eculizumab monoclonal antibody is administered by intravenous injection, and the drug is usually administered by intravenous injection with a short half-life. Therefore, by modifying the immunogenicity, the administration method can be transformed into a simpler and more convenient subcutaneous injection, which can realize self-administration in the patient's home and effectively improve the half-life of the drug in the body. There are no reports of low immunogenic C5 antibodies.

Antibody-dependent cell-mediated cytotoxicity (ADCC) refers to NK cells, macrophages and neutrophils that express IgG Fc receptors, through binding to the surface of target cells. The Fc region of IgG antibodies binds to and kills these target cells. IgG antibodies can mediate these cells to play the role of ADCC, among which NK cells are the main cells that can play the role of ADCC. In the process of antibody mediated ADCC, the antibody can only specifically bind to the corresponding epitope on the target cell, and effector cells such as NK cells can kill any target cell that has been bound to the antibody. The antigen binding on the cell is specific, and the killing effect of NK cells on target cells is non-specific.

CDC refers to the formation of MAC on the surface of target cells after the activation of the complement system, leading to the lysis of target cells, an effect called complement-dependent cytotoxicity. Complement can lead to the lysis of a variety of bacteria and other pathogenic organisms and is an important defense mechanism of the body against pathogenic organisms. It plays an important role in preventing the infection of Gram-negative bacteria. In some cases, the complement system can cause tissue or cell damage and be involved in the pathogenesis of hypersensitivity reactions and autoimmune diseases.

To avoid the unfavorable ADCC and CDC functions induced by the antibody binding to the target protein, the currently commonly adopted method is to glycosylation mutation of the 298 amino acid N to A in the heavy chain of the antibody, or to change the heavy chain of the antibody to IgG4 or IgG2 Subtype. The amino acid heavy chain structure of the original Eculizumab adopts the IgG2 subtype. However, IgG2 subtype antibodies are less stable in structure than IgG1 subtype antibodies, easy to form soluble multimers, significantly shortened half-life, and less mature in production process. This brings risks and inconvenience to the use of clinical patients, and at the same time, the drug half-life is relatively short, and the drug dose needs to be increased to make up for this defect during use. Based on this, if the constant region of the antibody can be modified without affecting the affinity and specificity of the antibody, the IgG2 subtype of the antibody can be changed to the IgG1 subtype while reducing the immunogenicity and ADCC/CDC function of the antibody. On the one hand, the stability and safety of the monoclonal antibody can be improved; on the other hand, the half-life of the drug can be increased, which can also play a role in improving the curative effect while reducing its dosage. At present, there is no report on reducing the immunogenicity and ADCC/CDC function of C5 antibody by changing the amino acid sequence.

### SUMMARY OF THE INVENTION

The first object of the present invention is to provide a kind of anti-C5 humanized monoclonal antibody with low immunogenicity and low ADCC/CDC function.

Another object of the present invention is to provide a method for preparing low immunogenicity, low ADCC/CDC therapeutic antibodies including humanized anti-C5 monoclonal antibodies.

The present invention utilizes commercial DNAStar™ software to evaluate the original amino acid sequence of Eculizumab monoclonal antibody of Alexion Company, and the result shows that the immunogenicity coefficient of Eculizumab is 18. The above software was used to evaluate the immunogenicity of the non-antigen-binding fragment (FR) in the variable region of the antibody and find the relevant sequences with strong immunogenicity. Subsequently, all FR segments involving light and heavy chains in the human antibody gene library were searched, and segments with high sequence homology and relatively low immunogenicity were selected after sequence alignment. The selected framework region was replaced with the corresponding segment in Eculizumab, and then 3D modeling was performed on the replaced sequence, and the structure was compared with the original research. The structure is analyzed, a relatively flexible region is found between the variable region and the constant region of the antibody, and a flexible amino acid sequence is inserted into the corresponding region, and the corresponding sequence is synthesized and sequenced, and the correct sequence is selected for subsequent functional confirmation.

Sequence the synthesized gene and select the correct sequence to proceed to the next step. The design restriction site of the light chain variable region is Hind III+EcoR I, and the design restriction site of the heavy chain variable region is Hind III+EcoR I, respectively connect with the expression vectors pEE12.4 (heavy chain) and pEE6.4 (light chain) and transform E. coli DH5α at the same time to obtain heavy chain and light chain chimeric antibody expression vectors. At the same time, the constructed antibodies were sequenced and sequenced; the plasmids were amplified for the above-expressed vectors, and Qiagen's endotoxin-free plasmid amplification kit was selected; the selected plasmids were optimized and combined, and CHO cells were used for transient transfection Expression, the expressed antibody is subjected to affinity and EC50 detection, and a combination is selected to construct a stable strain according to the detection result; according to the above detection result, the present invention selects a corresponding combination to construct a stable strain by electro transfection, and at the same time uses MTX to measure the degree of antibody expression. For the screening of the pressurized stable strains, monoclonal screening is carried out, and the stable strains with higher antibody production are finally selected.

In the present invention, while the structure of the antibody framework region is modified to reduce the immunogenicity of the antibody, it is also expected to reduce the ADCC/CDC function of the antibody. By inserting a flexible sequence into the constant region of the antibody heavy chain, the mechanical stress transmission generated after the antibody variable region binds to the antigen is cut off, so that the constant region of the antibody drug heavy chain cannot be fully exposed with Fc receptors and/or complement binding sites. Weakening the binding of antibody drugs to killer cells expressing IgG Fc receptors such as NK cells, macrophages and neutrophils, or to complement binding, cannot or reduce the signal that induces ADCC and CDC.

The invention also changes the antibody subtype from IgG2 to IgG1 based on the Eculizumab antibody drug of ALEXION Company and inserts a flexible amino acid sequence between the CDR3 and CH2 regions of the heavy chain of the IgG1 antibody to block the antibody from binding to the antigen. Stress transfer in variable and constant regions, thereby reducing ADCC/CDC function without significantly increasing multimer formation.

The anti-C5 humanized monoclonal antibody with low immunogenicity and low ADCC function of the present invention is the amino acid modification of the non-antigen-binding region in the original Eculizumab sequence to reduce its immunogenicity. The full length of the light chain and the original Eculizumab sequence are the amino acid sequences of the heavy chain variable regions are shown in SEQ ID NO. 1 and 2, respectively.

The anti-C5 humanized monoclonal antibody with low immunogenicity and low ADCC function of the present invention, its heavy chain variable region contains the amino acid sequence shown in SEQ ID NO.3 or 4 or the amino acid shown in SEQ ID NO.3 or 4 The amino acid sequence of the protein with the same function obtained by the replacement, deletion or insertion of one or more amino acids, and its light chain contains the amino acid sequence shown in SEQ ID NO.1 or the amino acid sequence shown in SEQ ID NO.1. The amino acid sequence of a protein with the same function is obtained by substitution, deletion, or insertion of one or more amino acids.

Further, the anti-C5 humanized monoclonal antibody with low immunogenicity and low ADCC function of the present invention contains the amino acid sequence shown in SEQ ID NO.5 or 6 or the amino acid sequence shown in SEQ ID NO.5 or 6 in its heavy chain The amino acid sequence of the protein with the same function obtained by the substitution, deletion or insertion of one or more amino acids, its light chain contains the amino acid sequence shown in SEQ ID NO.1 or the amino acid sequence shown in SEQ ID NO.1 through a The amino acid sequence of a protein with the same function obtained by substitution, deletion or insertion of multiple amino acids.

In the combination of the above-mentioned light chain and heavy chain sequences, the present invention obtains a plurality of monoclonal antibody sequences, which not only maintain the original binding affinity with human C5, can specifically block the C5 complement hemolytic activity and C5a production, but also The functional activities of ADCC and CDC are reduced, and since they are both IgG1 subtype monoclonal antibodies, the production process of the monoclonal antibody of the present invention is mature, the difficulty is low, and the anti-polymerization, expression and stability of the antibody are improved.

Specifically, the IgG1 subtype low ADCC/CDC functional C5 monoclonal antibody provided by the present invention has the full-length amino acid of the light chain as shown in L0 (SEQ ID NO. 1), and the full-length amino acid sequence of the heavy chain has H1 (SEQ ID NO. 1) . . . 5), H3 (SEQ ID NO.6) any sequence shown by these two nucleotide sequences.

The present invention provides genes encoding the above-mentioned monoclonal antibody light chain and heavy chain.

The variable region of the heavy chain of the gene contains the nucleotide sequence shown in SEQ ID NO.9 or 10, and the light chain of the gene contains the nucleotide sequence shown in SEQ ID NO.7.

The present invention provides an expression vector containing the above-mentioned IgG1 subtype low ADCC/CDC functional C5 monoclonal antibody light chain and heavy chain genes. Host bacteria, host cells or expression cassettes containing the expression vector are also within the protection scope of the present invention.

The present invention provides the application of the above-mentioned monoclonal antibody encoding gene or biological material containing the gene in the treatment of diseases targeting C5.

The present invention provides the application of the above-mentioned IgG1 subtype low ADCC/CDC functional C5 monoclonal antibody in the preparation of a medicine for treating diseases.

Said disease is tumor, immune function decline and so on.

The medicine is an anti-tumor medicine for treating paroxysmal nocturnal hemoglobinuria, atypical hemolytic uremic syndrome, glomerulonephritis, and immune complex-mediated nephropathy.

The present invention provides a drug or detection reagent containing the above-mentioned anti-C5 monoclonal antibody with low immunogenicity and low ADCC/CDC function of IgG1 subtype.

The present invention provides the application of the above-mentioned anti-C5 monoclonal antibody with low immunogenicity and low ADCC/CDC function of IgG1 subtype in the treatment of diseases targeting C5.

The humanized anti-C5 antibody and Eculizumab monoclonal antibody provided by the present invention target the same site of C5, but their immunogenicity is low and the three-dimensional structure and conformation of the original antibody are similar. With lower originality, fewer side effects (such as immune complex-mediated clearance of antibodies or fragments from circulation), longer half-life, and higher stability, it is expected to become a very ideal biological targeted therapeutic antibody. The present invention significantly reduces the risk of immunogenicity of the antibody drug in patients by modifying the constant region and variable region sequences of Eculizumab monoclonal antibody. The examples of the present invention show that the improved Eculizumab antibody of the present invention has similar binding affinity to C5 as the original Eculizumab, and significantly reduces the immunogenicity of the antibody drug, prolongs the half-life of the antibody drug, and improves the curative effect.

### DESCRIPTION OF DRAWINGS

FIG. 1 The result of double enzyme digestion of heavy chain and light chain of the modified monoclonal antibody of the present invention. Among them, a is the double-enzyme digestion result of the heavy chain H0 plasmid Hind III and EcoRI I of the monoclonal antibody of the Aiku strain, and the swimming lanes from left to right are: the plasmid before restriction digestion, the plasmid after restriction digestion and the DNA marker; b is provided by the present invention The heavy chain H1 plasmid Hind III and EcoRI double digestion results of the monoclonal antibody, the lanes from left to right are: plasmid before digestion, plasmid after digestion and DNA marker; c is the weight of the monoclonal antibody provided by the present invention. Chain H3 plasmid Hind III and EcoRI double digestion results, lanes from left to right are: plasmid before digestion, plasmid after digestion and DNA marker; d is the light chain L0 plasmid Hind III and EcoRI of Aiku monoclonal antibody The results of double digestion, the lanes from left to right are: plasmid before digestion, plasmid after digestion and DNA marker.

FIG. 2 results of the modified mAb affinity EC50 of the present invention.

FIG. 3 ADCC activity experiment of the modified monoclonal antibody of the present invention and the original Eculizumab antibody.

FIG. 4 Comparison of the ADA titer of the modified monoclonal antibody of the present invention and the original Eculizumab antibody in mice.

### DETAILED DESCRIPTION

Abbreviations and Definitions

“Antibody” refers to any form of antibody that exhibits a desired biological activity (eg, inhibition of binding of a ligand to its receptor or by inhibition of ligand-induced receptor signaling). Accordingly, such antibodies are used in the broadest sense and specifically include, but are not limited to, monoclonal, polyclonal, and multipolicy antibodies.

A “Fab fragment” consists of the CH1 and variable regions of one light chain and one heavy chain. The heavy chain of a Fab molecule cannot form a disulfide bond with another heavy chain molecule.

The “Fc” region is the two heavy chain fragments containing the CH1 and CH2 domains of an antibody. The two heavy chain fragments are held together by two or more disulfide bonds and by hydrophobic interactions of the CH3 domains.

A “Fab′ fragment” contains a light chain and a portion of a heavy chain comprising the VH and CH1 domains and the region between the CH1 and CH2 domains, thus allowing between the two heavy chains of two Fab′ fragments Interchain disulfide bonds are formed to form F(ab′)2 molecules.

The term “monoclonal antibody” as used herein refers to an antibody obtained from a population of substantially homogeneous antibodies, i.e. the individual antibodies comprising the population are identical except for possible natural mutations that may be present in minor amounts. Monoclonal antibodies are highly specific and can be directed against a single antigenic site. Furthermore, each monoclonal antibody is directed against only a single determinant on an antigen, as opposed to conventional (polyclonal) antibody preparations, which typically include multiple different antibodies directed against multiple distinct determinants (epitopes). The modifier “monoclonal” denotes the property of an antibody obtained from a substantially homogeneous population of antibodies and should not be construed as requiring any method for the preparation of said antibody.

The term “immune cells” as used herein includes cells of hematopoietic origin and that play a role in immune responses. Immune cells include lymphocytes, such as lymphoid B cells and lymphoid T cells; natural killer cells; myeloid cells, such as monocytes, macrophages, eosinophils, mast cells, basophils, and granulocytes.

The following nucleic acid puns are used herein: R=A or G; Y=C or T; M=A or C; K=(G or T); S=G or C; and W=A or T.

As used herein, the term “about” refers to an index value that is within an acceptable error range of the value determined by one of ordinary skill in the art, which value depends in part on how it is measured or determined (i.e., the limits of the measurement system). For example, “about” can mean within 1 or more than 1 standard deviation in every practice in the art. Alternatively, “about” or “substantially comprising” can mean a range of up to 20%. Also, with respect to a biological system or process, the term can mean up to an order of magnitude or up to 5 times the value. Unless otherwise stated, when a specific value appears in this application and in the claims, the meaning of “about” or “substantially comprising” is assumed to be within an acceptable error range of the specific value.

“Specific” binding when referring to a ligand/receptor, antibody/antigen or other binding pair refers to determining the presence or absence of binding of a protein such as PD-1 in a heterogeneous population of proteins and/or other biological agents' reaction. Thus, under the specified conditions, a specific ligand/antigen binds to a specific receptor/antibody and does not bind to other proteins present in the sample in significant amounts.

When using “administer” and “treat” in reference to an animal, human, subject, cell, tissue, organ, or biological fluid, it refers to combining an exogenous drug, therapeutic agent, diagnostic agent or composition with the animal, human, subject, or biological fluid. Person, cell, tissue, organ, or biological fluid contact. “Administering” and “treatment” can refer to, for example, therapeutic methods, pharmacokinetic methods, diagnostic methods, research methods, and experimental methods. Treating the cells includes contacting the agent with the cells and contacting the agent with a fluid, wherein the fluid is in contact with the cells. “Administering” and “treating” also mean in vitro and ex vivo treatment of cells, e.g., by agents, diagnostic agents, binding compositions, or by other cells.

“Inhibit” or “treat” or “treatment” as used herein includes delaying the development of symptoms associated with a disease and/or reducing the severity of those symptoms that will or are expected to develop in the disease. The term also includes alleviation of existing symptoms, prevention of additional symptoms, and alleviation or prevention of underlying causes of such symptoms. Thus, the term indicates that a beneficial outcome has been conferred on a vertebrate subject suffering from a disease.

The therapeutic applications of the antibodies of the present invention are as follows:
I. Immune Complex Mediated Nephropathy

The formation of immune complexes is typically the result of the interaction of antigens with specific antibodies. The inflammatory response that occurs when this complex accumulates in a limited area is an important element of normal host defense, leading to clearance of immune complexes and antigen destruction by phagocytes. In contrast, immune-complex-related diseases arise from excessive complex formation or delayed clearance, usually in the context of specific antigenic challenge or immune dysregulation. In this case, immune complexes form and deposit at specific tissue sites, triggering an inflammatory response and leading to local or systemic tissue damage. The kidneys, especially the glomerular structures, are highly susceptible sites for immune complex deposition during severe disease development.

Human studies and studies using animal models of human disease have implicated the complement system in pathologies associated with many immune complex-related diseases. Activation of complement that mediates the pathology associated with these conditions may be the result of autoimmune mechanisms or may be of non-immunogenic origin.

A hypersensitivity reaction that occurs after an antibody binds to an antigen present in a tissue or circulatory system, which is caused by the activation of complement and the release of molecules that mediate inflammation. This process is mediated by the binding of antibodies to fixed tissue, cell-bound antigens (type II hypersensitivity), or circulating antigens, further leading to the formation of circulating immune complexes and their pathogenic deposition in tissues (type III hypersensitivity reactions) reaction).

After binding of the antibody to the fixed tissue antigen, it mediates type II hypersensitivity by activating complement. Subsequent activation of proinflammatory and lytic components of the complement system recruits stimulated leukocytes to sites of immune complex formation, further eliciting an inflammatory response. At the same time, the anaphylactic toxicity of C3a and C5a caused increased vascular permeability, which further enhanced immune complex deposition and leukocyte recruitment.

Cross-linking of antibody-bound cells or tissues to effector cells (eg, neutrophils, platelets, N cells, and monocytes) through their Fc receptors can also lead to pro-inflammatory effects. This cross-linking activation of effector cells can stimulate the release of oxygen free radicals, prostaglandins and leukotrienes, and the release of these three components will further activate the action of complement components.

Type II hypersensitivity-mediated conditions include hyperacute rejection of transplanted organs, autoimmune hemolysis and thrombocytopenia, Goodpasture syndrome (and other associated glomerulonephritis and pulmonary hemorrhage), myasthenia gravis, insulin-dependent Diabetes-related pathological sequelae and pemphigus vulgaris.

Type III hypersensitivity reactions involving circulating antigens can also lead to the development of many pathological conditions. These include glomerulonephritis (discussed in detail below), vasculitis (a potentially life-threatening inflammation of large and/or small blood vessels), rheumatoid arthritis, dermatitis, and other conditions. Other diseases associated with type III hypersensitivity reactions include autoimmune diseases such as systemic lupus erythematosus (SLE), various types of infectious diseases, tumors, and a variety of other conditions.
II. Paroxysmal Nocturnal Hemoglobinuria

Paroxysmal nocturnal hemoglobinuria (PNH) is a non-malignant clone caused by an acquired somatic PIG-A (phosphatidyl inositol glycan complementation group A) mutation in one or several hematopoietic stem cells Sexual diseases, PIG-A mutation causes abnormal synthesis of glycosyl phosphatidyl inositol (GPI), resulting in the loss of a group of membrane proteins anchored on the cell membrane by GPI, including CD16, CD55, CD59, etc. Manifested as chronic intravascular hemolysis, hematopoietic failure, and repeated thrombosis.

Typical PNH is characterized by chronic intravascular hemolysis, hemoglobinuria, and hemosiderin. The peak age of onset is between 20 and 40 years old, and individual cases occur in children or the elderly, and males are significantly more than females. In a domestic summary of 203 PNH patients, the first symptom was anemia in 56.7%, hemoglobinuria in only 12.8%, and jaundice and anemia in 5.9%.

Conventional treatment is mainly to control hemolytic episodes, such as dextran, sodium bicarbonate, adrenocortical hormones and other immunosuppressive agents, as well as androgen stimulation of hematopoiesis. Recently, PNH treatment mainly includes: Eulizumab, combination chemotherapy, allogeneic hematopoietic stem cell transplantation, and anticoagulation therapy.
III. Atypical Hemolytic-Uremic Syndrome

Atypical haemolytic uraemic syndrome (aHUS) is a complement disorder in which mutations in the complement regulatory protein factor H, as well as membrane accessory proteins and serum complement intrinsic components (factor B, complement C3) are involved. The onset of the disease is easy to repeat, and the prognosis is very poor. 25% of patients die in the acute phase, and more than 50% develop end-stage renal disease.

Long-term activation of complement damages cells in the body that lack complement-inhibiting factors, leading to an inflammatory response throughout the circulatory system. The endothelial cells on the inner side of the vascular lumen are damaged and swollen, and neutrophils and other inflammatory cells will accumulate to the damaged part (vascular endothelial cells), causing inflammation of small blood vessels. Platelets lacking complement inhibitors are also directly activated by complement, resulting in widespread multiple thrombosis throughout the vasculature.

Blood clots and inflammatory reactions block blood flow in the body's blood vessels, reducing blood supply to organs and cells, resulting in a state of hypoxia that leads to damage and failure of organs, including the brain, kidneys, heart, and gastrointestinal tract. Summary There are three main features of atypical hemolytic uremic syndrome: microvascular hemolytic anemia, thrombocytopenia, and renal failure.
IV. Glomerulonephritis

Glomeruli are key structural and functional elements that make up the kidney. A single glomerulus, called a nephron, is the main functional unit of the kidney. Each kidney has about one million nephrons. Each glomerulus consists of a network of up to 50 parallel capillaries enclosed in the structure of Bowman's capsule. The space within Bowman's capsule that is not occupied by the glomerular capillary absorptive domain network is called Bowman's space. The glomerulus acts as a filter, separating water and specific solutes from proteins and cells in the blood into Bowman's space for further processing.

Glomerulonephritis (GN) is a disease caused by the accumulation of immune complexes. The accumulation of immune complexes in the glomerulus leads to an inflammatory response and cell proliferation, which in turn leads to narrowing of the capillary lumen and partial or complete obstruction of the glomerulus. One consequence of this process is the inhibition of the normal filtering function of the glomeruli. Obstruction can occur in a large number of glomeruli, can directly impair renal function and predispose to deposition of proteins in the glomerular capillary walls. This deposition in turn leads to damage to the glomerular basement membrane. On the other hand, the permeability of those unobstructed glomeruli will increase, causing large amounts of protein to pass into the urine, a condition known as proteinuria.

In many severe cases of GN, pathological structures called crescents form within Bowman's space, which further impede glomerular filtration. These structures can only be observed through tissue samples and are therefore not observed in all patients. Crescents are a manifestation of hypercellularity and are thought to arise from extensive abnormal proliferation of parietal epithelial cells (the cells that form the lining of Bowman's capsule). Clinical studies have shown a correlation between the percentage of glomeruli and crescents and the clinical severity of the disease. The presence of large numbers of crescents is a marker of poor prognosis.

Symptoms of GN include proteinuria, decreased glomerular filtration rate (GFR), changes in serum electrolytes including azotemia (uremia, excess blood urea nitrogen-BUN), and salt retention. GN can lead to hypertension and edema due to water retention, hematuria, and abnormal urinary sedimentation (including erythrocyte casts, hypoalbuminemia, hyperlipidemia, and lipuria).

The present invention will be more fully understood by reference to the following examples. However, these examples should not be construed as limiting the scope of the present invention. All literature and patent citations mentioned herein are expressly incorporated herein by reference.

Unless otherwise specified, the technical means used in the examples are conventional means well known to those skilled in the art. The materials, reagents, etc. used in the following examples can be obtained from commercial sources unless otherwise specified.

### EXAMPLE 1

### Analysis and Design of Eculizumab mAbs with Low ADCC/CDC Function to Reduce Immunogenicity

The original sequence of ALEXION's Eculizumab was evaluated by commercial DNAStar™ software, and the results showed that the immunogenicity coefficient of Eculizumab antibody drug was 18. Find a relatively flexible region between the antibody variable region and the constant region, and insert a flexible amino acid sequence into the corresponding region to cut off the mechanical stress transmission generated after the antibody variable region binds to the antigen, so that the heavy chain constant region of the antibody drug can interact with the Fc receptor. And/or the complement binding site cannot be fully exposed, weakening the binding of antibody drugs to killer cells expressing IgG Fc receptors such as NK cells, macrophages, and neutrophils or to complement binding, which cannot or reduces the induction of ADCC and CDC. signal of.

Specifically, the constant region of ALEXION's Eculizumab antibody drug was modified, the antibody subtype was changed from IgG2 to IgG1, and a flexible amino acid sequence was inserted between the CDR3 and CH2 regions of the heavy chain of the antibody to block the antibody from binding to the antigen. Stress transfer in variable and constant regions, thereby reducing ADCC/CDC function without significantly increasing multimer formation. The full length of the light chain and the amino acid sequence of the variable region of the heavy chain of the original Eculizumab sequence are shown in SEQ ID NO. 1 and 2, respectively.

The anti-C5 humanized monoclonal antibody with low immunogenicity and low ADCC/CDC function provided by the present invention has the amino acid sequence of the heavy chain shown in SEQ ID NO. 5 and 6, and the amino acid sequence of the full length of the light chain is SEQ ID NO.1, named H1L0 and H3L0 respectively.

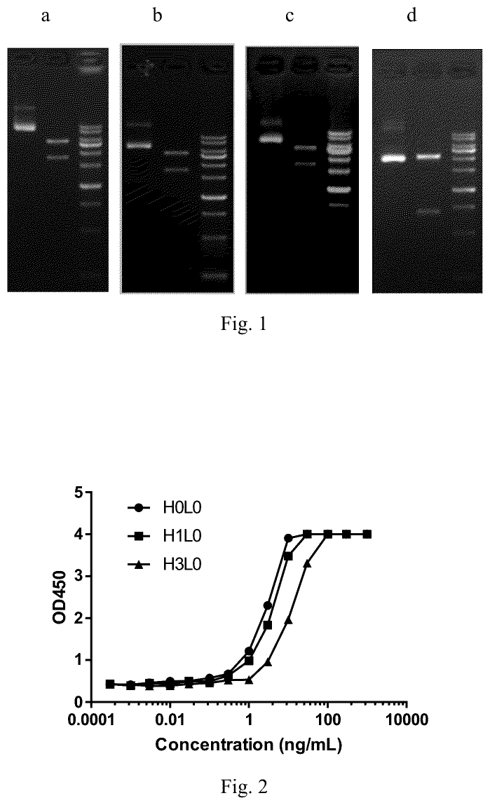

According to the known antibody heavy chain glycosylation information, the present invention performs whole gene synthesis on the corresponding modified sequence, sequenced the synthesized gene, and selects the sequenced correct sequence for the next operation, and designs the enzyme in the light chain variable region. The cleavage site is Hind III+EcoR I, and the heavy chain variable region design enzyme cleavage site is Hind III+EcoR I, which are respectively connected to the expression vectors pEE12.4 (heavy chain) and pEE6.4 (light chain) vectors, and at the same time Escherichia coli DH5α was transformed to obtain heavy chain and light chain chimeric antibody expression vectors. At the same time, the constructed antibodies were sequenced and sequenced; the plasmids were amplified for the above-expressed vectors, and Qiagen's endotoxin-free plasmid amplification kit was selected; the selected plasmids were optimized and combined, and CH0 cells were used for transient transfection According to the detection results, which combination is selected for stable transfection; according to the detection results, the present invention selects the corresponding combination to construct stable strains by electro transfection, and at the same time uses MTX screened the degree of antibody expression and monoclonal screening of the pressurized stable strains, and finally selected stable strains with higher antibody yields for use in subsequent experiments. The affinity ELISA results of selecting H1L0, H3L0 and C5 showed that the affinity of H1L0, H3L0 and C5 was comparable to that of the original research drug Eculizumab antibody, as shown in FIG. 2.

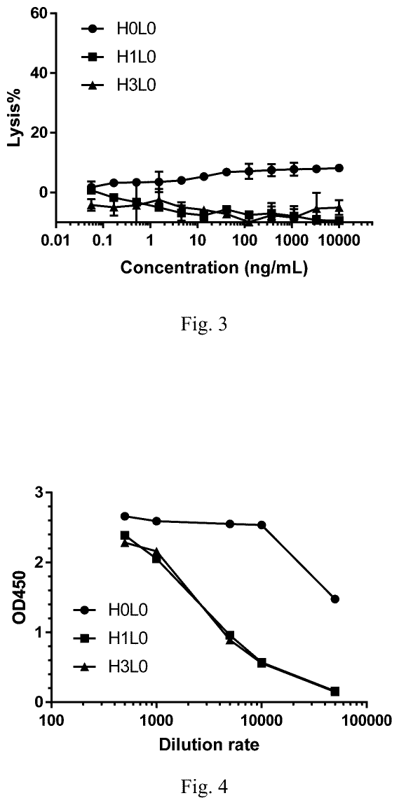

The ADCC activity experiment of H1L0, H3L0 and the original research drug Eculizumab antibody showed that the two monoclonal antibodies H1L0, H3L0 of the present invention and the original research drug Eculizumab antibody had no ADCC activity, as shown in FIG. 3.

H1L0, H3L0 and the original research drug Eculizumab antibody ADA titer comparison experiment, mice were selected 7 days, 14 days, and 21 days after immunization for tail blood collection and detection, and the results are shown in FIG. 4. The results show that the immune response of the monoclonal antibody produced by the present invention in mice is significantly lower than that of Eculizumab, especially H1L0 is the lowest.

It can be seen that two monoclonal antibody sequences are obtained by screening in the present invention, which not only maintains the original binding affinity with human C5 but can specifically block the binding of C3 and C5, and at the same time, the immunogenicity is effectively reduced. The results of the ADCC activity verification show that the ADCC activity of the two monoclonal antibody sequences obtained by the screening of the present invention and the original Eculizumab have no ADCC function.

### EXAMPLE 2

### Construction of an Improved Eculizumab mAb Expression Vector

According to the modified Eculizumab heavy chain and light chain variable region base sequences obtained by sequencing in Example 1, the restriction sites on both sides of the light chain sequence were designed as Hind III+EcoR I, and the restriction sites on both sides of the heavy chain sequence were designed The point is Hind III+EcoR I. The above sequence was sent to Jinweizhi Company to synthesize the whole gene sequence, and pEE12.4 (heavy chain) and pEE6.4 (light chain) were used as expression vectors. After the synthesis of the gene, double-enzyme digestion verification was carried out. (See FIG. 1), and the corresponding strains were subjected to plasmid extraction and sequence determination. The sequencing results showed that the sequences of the two were completely identical, indicating that the antibody expression vector was successfully constructed.

### EXAMPLE 3

### Transient Expression and Purification of Improved Eculizumab mAb

Escherichia coli DH5α was transfected with the pEE12.4 heavy chain and pEE6.4 light chain expression vectors constructed in Example 2. It was inoculated into 100 mL of LB medium and cultured according to conventional methods. The cultures were harvested, and plasmid DNA was extracted and purified using the UltraPure Plasmid DNA Homozygosity Kit from Qiagen. The purified plasmid DNA was transfected into 293F cells using the liposome method kit from Invitrogen, and the operation was carried out according to the manufacturer's instructions.

First, 293F cells were transfected with different combinations of light and heavy chain plasmids. The combinations are shown in Table 1. There are 3 groups of 293F transiently expressed, and the combination with reduced immunogenicity needs to be screened from these 3 groups. After culturing for 3 days, the culture supernatant was taken for antibody expression detection. The results are shown in Table 1 below:

TABLE 1
Transient antibody expression levels

| Combination | Expression mg/L |
|---|---|
| H0L0 (Original) | 7.34 |
| H1L0 (Improved) | 9.94 |
| H3L0 (Improved) | 6.21 |

### EXAMPLE 4

### Determination of Biological Activity of the Improved Eculizumab Monoclonal Antibody

1. Affinity Evaluation

This part uses ELISA indirect method to measure antibody EC50 to evaluate antibody affinity.

The experimental method is as follows: Dilute the antigen with C5 (purchased from Nearshore Technology Co., Ltd.) to 0.3 μg/ml with PBS; add 100 μl/well of the diluted antigen to a 96-well plate, cover, overnight at 4° C.; shake off The liquid in the well was washed three times with PBS, 200 μl/well, and patted dry; blocked with 5% milk-PBS 200 μl/well for 1 hour, and tapped every 15 minutes; the liquid in the well was shaken off, washed once with PBS, 200 μl/well, and patted dry; Add purified antibody (0-10 μg/ml) in a gradient, see Table 4 for antibody name, 5% milk-PBS dilution, 100 μl/well, incubate for 1 h, tap every 15 min; shake off the liquid in the well, wash three times with PBS, 200 μl/well, pat dry; add secondary antibody, diluted in 5% milk-PBS, 100 μl/well, incubate for 1 h, tap every 15 min; preheat TMB substrate at room temperature, and turn on the microplate reader to preheat; wash 5 times with PBS, 250μl/well, the first three times 5 min, the last two times 10 min, pat dry; add 50 μl/well of TMB substrate AB each, and develop color at room temperature for 20 min; use a scanner to scan and record the picture, add 50 μl/well of stop solution, use enzyme labeling The meter reads OD450. The experimental results are shown in FIG. 2. The EC50 of the improved Eculizumab mAb was calculated from the experimental data, and the results are shown in Table 2.

TABLE 2
Affinity evaluation results of improved Eculizumab mAb

| | Original H0L0 | Improved H1L0 | Improved H3L0 |
|---|---|---|---|
| EC50 (ng/mL) | 2.478 | 3.824 | 11.46 |
| EC50 (M) | 1.652E−11 | 2.55E−11 | 7.64E−11 |

2. Specificity Evaluation

This process verifies whether the expressed antibody is specific for C5, using different factors to coat the plate and use an indirect method.

The specific experimental process is as follows: C5, C3, C3b, rIFN γ (recombinant human interferon γ), IL-1α, IL-1β, IL-2, IL-4 and IL-8 (purchased from Inshore Technology, respectively) were mixed with PBS. Co., Ltd.), dilute the antigen to 1 μg/ml; add 100 μl/well of the diluted antigen to a 96-well plate, cover, overnight at 4° C.; shake off the liquid in the well, wash three times with PBS, 200 μl/well, manually Pat dry; block with 200 μl/well of 5% milk, block for 1 h, tap the edge of the ELISA plate every 15 minutes to promote the reaction; shake off the liquid in the well, wash once with PBS, 200 μl/well, pat dry by hand; add different light chains Heavy chain combination antibody, diluted in 5% milk, 100 μl/well, incubated for 1 h, tap the edge of the ELISA plate every 15 min to promote the reaction; shake off the liquid in the well, wash three times with PBS, 200 μl/well, pat dry by hand; add Goat anti-human secondary antibody, diluted in 5% milk, 100 μl/well; incubate for 1 h, tap the edge of the ELISA plate every 15 min to promote the reaction; preheat the TMB substrate at room temperature, and turn on the microplate reader to preheat; wash 5 times with PBS, 250 μl/well, the first three times 5 min, the last two times 10 min, pat dry by hand; add 50 μl/well of TMB substrate AB to each well, and develop color at room temperature for 20 min; use a scanner to scan and record the pictures, add 50 μl/well of stop solution, use enzyme The standard instrument reads OD450 and archives. The experimental results are shown in Table 3:

TABLE 3
Specificity analysis of improved eculizumab

1. Coating factor

| Ab | Improved H1L0 | Improved H3L0 |
|---|---|---|
| C5 | + | + |
| C3 | − | − |
| C3b | − | − |
| rIFNγ | − | − |
| IL-1α | − | − |
| IL-1β | − | − |
| IL-2 | − | − |
| IL-4 | − | − |
| IL-8 | − | − |
| NC | − | − |

Note:
“+” means to identify the corresponding coating factor, “−” means not to identify the corresponding coating factor.

The above results show that the improved Eculizumab is specific for C5

### EXAMPLE 5

### Evaluation of ADCC Function of Improved Eculizumab Monoclonal Antibody

In order to evaluate the ADCC function of the antibody in the present invention, the present invention evaluates the ADCC activity of the antibody by detecting the killing ability of FcR-TANK cells to cells overexpressing the target protein (C5).

After using the target protein overexpressing cells to be stained with CFSE, the cell density was adjusted to 6×105/mL, and the modified antibodies (H1L0, H3L0) and Eculizumab were mixed in a certain proportion gradient (the highest concentration was 4 μg/mL, and the medium was used three times) Dilute 12 gradients) for dilution, at the same time count FcR-TANK cells and adjust the cell density to 6×105/mL; co-incubate antibody, target cells and effector cells: take 50 μL of each of the above-mentioned different dilution gradients, target 50 μL of cells and 100 μL of FcR-TANK cells were added to a 96-well plate, and each gradient was subjected to repeated well operation, and a blank control was set at the same time (150 μL of medium+50 μL of target cell agent medium 50 μL+50 μL of target cells+100 μL of FcR-TANK cells). Incubate for 4 h at 37° C., 5% CO2. After incubation, place the cell culture plate at room temperature for 10 min, add PI staining solution (final concentration of 5 μg/mL) and mix well. Flow cytometry was used to analyze the positive rate of PI staining of cells at different concentrations, and then the antibody mediated ADCC intensity was calculated. The calculation formula was ADCC %=(Sample % PI Positive Cell−No Antibody % PI Positive Cell)/(100−No Antibody % PI Positive Cell)×100, and plot the relationship between ADCC % and concentration, as shown in FIG. 3.

The modified C5 monoclonal antibody of the present invention and the original research drug Eculizumab antibody ADCC activity experiment showed that the two monoclonal antibodies H1L0 and H3L0 of the present invention both proved that the ADCC effect was completely removed, as shown in FIG. 3.

### EXAMPLE 6

### Evaluation of the Immunogenicity of the Improved Eculizumab Monoclonal Antibody

Mouse Immunization Experiment
1) Basic immunization: The antigen and Freund's complete adjuvant were mixed with equal volume and fully emulsified, and subcutaneously injected at points, and the injection amount was 100 μg per Balb/c mouse.
2) Booster immunization: Emulsion of antigen and incomplete Freund's adjuvant is used for booster immunization.

After the above experiments were completed, an ELISA detection test was carried out, and the results were shown in FIG. 4.

The above results show that the immunogenicity of the two improved Eculizumab mAbs of the present invention has been effectively reduced compared with the original Eculizumab mAb.

### INDUSTRIAL APPLICABILITY

The invention provides an anti-C5 humanized monoclonal antibody with low immunogenicity and low ADCC/CDC function and application thereof. By modifying the amino acid sequence of the framework region of Eculizumab monoclonal antibody, the immunogenicity was reduced, and the antibody was replaced from IgG2 subtype to IgG1 subtype, and a flexible amino acid sequence was inserted between the CDR3 and CH2 regions of the heavy chain of the IgG1 antibody to reduce the immunogenicity. The purpose of ADCC/CDC function is to improve the stability of the antibody and prolong its half-life. The binding affinity of the monoclonal antibody of the invention to human C5 is similar to that of the original Eculizumab antibody, it can specifically block the complement hemolytic activity of C5 and the production of C5a, and can be used for the preparation of C5-targeted paroxysmal nocturnal hemoglobinuria and atypical The therapeutic drug for hemolytic uremic syndrome has excellent clinical therapeutic value and application prospect.

What is claimed is:

1. an anti-C5 humanized monoclonal antibody with low immunogenicity and low ADCC/CDC function, is characterized in that, the amino acid sequence of the original Eculizumab monoclonal antibody light chain is shown in SEQ ID NO.1 and the heavy chain variable region of the original Eculizumab monoclonal antibody amino acid sequence (shown in SEQ ID NO.2) is remodeled by replacing the Fc of IgG2 with the Fc of IgG1, and a flexible amino acid sequence is inserted between the CDR3 and CH2 regions of the heavy chain of the IgG1 antibody.

2. The low immunogenicity/low ADCC/CDC anti-C5 humanized monoclonal antibody as claimed in claim 1, is characterized in that, the flexible amino acid sequence comprises GGGS, GGGSGGGS, GGSGGS.

3. The low immunogenicity/low ADCC/CDC anti-C5 humanized monoclonal antibody as claimed in claim 1, is characterized in that, its heavy chain variable region contains amino acidic sequences shown in SEQ ID NO.3 or 4, and its light chain contains the amino acid sequence shown in SEQ ID NO.2.

4. The low immunogenicity/low ADCC/CDC anti-C5 humanized monoclonal antibody as claimed in claim 1˜3, it is characterized in that its heavy chain contains the amino acid sequence shown in SEQ ID NO.5 or 6, and its light chain contains the amino acid sequence shown in SEQ ID NO.1.

5. The gene encoding the anti-C5 humanized monoclonal antibody of any one of claims 1 to 3.

6. gene as claimed in claim 5 is characterized in that, its heavy chain variable region contains the nucleotide sequence described in SEQ ID NO.9 or 10, and its light chain contains the nucleus shown in SEQ ID NO.7 nucleotide sequence.

7. The biological material containing the gene of claim 5 or 6, the biological material is an expression cassette, an expression vector, an engineered bacterium, or a cell.

8. the application of the anti-C5 humanized monoclonal antibody described in any one of claims 1 to 4, the gene described in claim 5 or 6 or the biological material described in claim 7 in the preparation of a medicine for treating a disease with C5 as a target.

9. the application of the anti-C5 humanized monoclonal antibody described in any one of claim 1˜4, the gene described in claim 5 or 6 or the biological material described in claim 7 in the preparation of medicine, and described medicine is treatment Drugs for paroxysmal nocturnal hemoglobinuria, atypical hemolytic uremic syndrome, glomerulonephritis, immune complex-mediated nephropathy.

10. The drug or detection reagent containing the anti-C5 humanized monoclonal antibody described in any one of claims 1 to 4.
